(19) United States
(12) Patent Application Publication
(10) Pub. No.: US 2021/0140958 A1
(43) Pub. Date: May 13, 2021

(54) BREVETOXIN ASSAY DEVICE, SYSTEM, AND METHOD

(71) Applicant: MOTE MARINE LABORATORY, INC., SARASOTA, FL (US)

(72) Inventors: Dana L. Wetzel, Sarasota, FL (US); Tracy Sherwood, Sarasota, FL (US)

(21) Appl. No.: 17/096,629

(22) Filed: Nov. 12, 2020

Related U.S. Application Data
(60) Provisional application No. 62/991,847, filed on Mar. 19, 2020.
(60) Provisional application No. 62/934,351, filed on Nov. 12, 2019.

(51) Int. Cl. G01N 33/543; C07K 16/14; G01N 33/53

(52) U.S. Cl. CPC G01N 33/54393; C07K 16/14; G01N 2333/405; C07K 2317/33; G01N 33/5308

(57) ABSTRACT

Brevetoxin (BTX) concentration levels in organisms and other samples are detected using conjugated recombinant monoclonal brevetoxin antibodies (BTX-rAbs) that are cross-reactive with one or more brevetoxin antigens. The analysis may employ a simplified and less pure, but more easily, more safely or more rapidly prepared crude tissue extract. The results may be used to permit or not permit the harvesting, sale, relocation or depuration of such organisms or samples.

### CROSS-REFERENCE TO RELATED APPLICATIONS

This application claims the benefit of U.S. Provisional Patent Application No. 62/934,351 filed Nov. 12, 2019 and entitled BREVETOXIN DETECTION DEVICE AND METHOD and U.S. Provisional Patent Application No. 62/991,847 filed Mar. 19, 2020 and entitled BREVETOXIN ASSAY DEVICE, SYSTEM, AND METHOD, the disclosures of both of which are incorporated herein by reference.

### TECHNICAL FIELD

This invention relates to methods and materials for testing samples for the presence and amount of brevetoxin.

### BACKGROUND

Red tide algal blooms are caused by Karenia brevis, an algal species that produces several brevetoxin (BT) compounds including both parent BT compounds and metabolite BT species (collectively, BTXs). Although most prevalent along the southwest Florida coast, and sometimes lasting over a year, red tide blooms have occurred along the entire US and Mexico Gulf coasts, and along the Atlantic coast as far north as North Carolina. BTXs are neurotoxic to a wide variety of organisms. Human consumption of bivalve mollusks (e.g., clams, oysters, mussels and scallops) containing sufficiently high BTX levels can lead to neurotoxic shellfish poisoning (NSP). Though BTXs tend to accumulate most significantly in shellfish, contamination of other marine organisms also commonly occurs.

K. brevis are lysed by wave action, particularly during blooms due the increase in the population density, causing the toxins to enter the water and then become aerosolized as sea spray. Aerosolized brevetoxins can be carried onshore by sea spray and produce respiratory distress among beachgoers and coastal residents. Exposure to the aerosolized toxins may result in eye and throat irritation, nasal congestion, cough, wheezing, shortness of breath, and further complications in individuals with chronic inflammatory lung conditions. Once airborne, aerosolized brevetoxins may further contaminate inland waters, crops, and other susceptible substances, substrates, and sites.

Conventional instrumented detection approaches for determining the danger posed by a red tide to beachgoers tended to offer insufficiently reliable or insufficiently timely measurement results. For the last 13 years, current best practice has relied on lifeguards to take periodic counts of how much coughing can be heard, as further described in U.S. Pat. No. 7,792,109 B2.

Three conventional protocols are commonly used for the assessment of NSP toxins in shellfish samples: a) the mouse bioassay (MBA), b) enzyme linked immunoassay (ELISA), and c) high performance liquid chromatography paired with a mass spectrophotometry detector (LC/MS). These protocols typically start with a shellfish tissue extract. The assays (and the governing regulations that may call for their use) focus on toxins in (or the potential toxicity of) the shellfish tissue, not on toxins in the surrounding water.

Water and other liquid sample (e.g., urine sample) preparation for evaluating BTX levels typically involves at least pretreatment to avoid absorption of the toxin by a glass sample container, and may also require blowdown or dilution to bring toxin concentration into a measurable range.

The National Shellfish Sanitation Program (NSSP) is a Federal/State cooperative program recognized by the U.S. Food and Drug Administration (FDA) and the Interstate Shellfish Sanitation Conference (ISSC) for the sanitary control of shellfish produced and sold for human consumption. In Florida, the Division of Aquaculture within the Florida Department of Agriculture and Consumer Services (FDACS) is responsible for the oversight, monitoring and regulation of NSSP guidelines for harvesting shellfish during a red tide. The assigned regulatory personnel perform periodic sampling and monitoring, usually on a weekly basis, in defined Shellfish Harvest Areas (SHAs) during red tide blooms. SHAs are closed when K. brevis cell counts reach or exceed 5,000 cells/L of seawater and are not reopened until the cell counts fall below that threshold of cells, or until the MBA is ≤20 MUs (mouse units or “MUs”) per 100 g of shellfish tissue, or until ELISA results are below BTX threshold limits set for clam or oyster ELISA results. If a farmer chooses to have samples analyzed via ELISA and the results exceed the threshold, then a follow-up analysis using the MBA is mandated. Because toxin exposure in an SHA can vary considerably over a short time period, there is a risk that in the intervening time period between sample collection and the receipt of results, SHA BTX concentrations could increase and contaminate bivalve shellfish that had tested safe only days before. In order to use the MBA with greater safety, regulators have adopted a series of complex and potentially redundant water and tissue testing protocols that reconcile the MBA time lag with the dynamic nature of K. brevis blooms.

Bivalves in a closed area can cleanse themselves of harmful BTX levels by a process known as depuration. Current practice in Florida and elsewhere in the United States during a red tide bloom is to allow bivalves to depurate naturally in closed areas after a red tide bloom has subsided. While this practice is effective in the absence of a prolonged red tide bloom, it can be highly problematic if blooms persist. For example, during the 2017-18 red tide bloom in southwest Florida, bivalve farms in Pine Island Sound and Gasparilla Sound were shut down for almost one year. Further, given the highly stochastic nature of red tide blooms, leaving bivalves in-situ during depuration can create considerable uncertainty for regulators tasked with supervising the process of reopening lease areas. One potential solution to this problem is to depurate bivalves affected by red tide in a controlled, alternate location. A number of recent studies have demonstrated that depuration of both clams and oysters is theoretically possible and technically feasible. Historically, however, depuration has been used only for purifying microbial contaminants from bivalves, and at present, there are no federally approved protocols for depurating BTXs from bivalves.

There consequently remains an unmet need for more effective and efficient systems and methods for assessing BTX levels in public waters, bivalve farms and other harvest areas. There further remains an unmet need for a reliable means of assessing BTX levels across the wide variety of sites, organisms, and food sources that can become contaminated. There also remains an unmet need for more effective and efficient systems and methods for assessing BTX levels in bivalves and other organisms, as well as water, urine, saliva, and food samples, as well as other sources and subjects of contamination.

### SUMMARY

The shellfish sample for an MBA is prepared by worldwide consensus using a protocol established by the American Public Health Association (APHA). For other shellfish assays, extracts typically are prepared using an acetone/methanol extraction method. Both the APHA method and the acetone/methanol extraction method employ an evaporation step, using for example a rotary evaporator. Typical steps for preparing extracts for MBA and ELISA analyses are described by Dickie et al., Multi-Laboratory Study of Five Methods for the Determination of Brevetoxins in Shellfish Tissue Extracts, Harmful Algae 2002, 10, pp. 300-302 (2004) as follows:
“Extracts were prepared using tissue homogenates of shellfish collected from Mobile Bay, Ala. (non-toxic oysters), and from Appalachicola Bay, Fla. during a 2001 K. brevis red tide (toxic oysters). Homogenates (100 g) for mouse bioassays were acidified with 1 mL 1N HCl, heated to boiling for 5 min, and extracted in diethyl ether using the American Public Health Association protocol specified for regulatory acceptability (Subcommittee on Laboratory Methods for the Examination of Shellfish, 1970). Diethyl ether was removed from the extracts by rotary evaporation, and the residues were suspended in saline containing 1% Tween-60 to achieve 10 mL total volume for mouse bioassays. Homogenates for the alternative methods were extracted in acetone (2:1 v/w). Solvent was removed from the extracts by rotary evaporation and the dried residues were re-solubilized in 80% methanol. Methanolic solutions were defatted with n-hexanes (1:1 v/v), evaporated, and resolubilized in 25% methanol. These methanolic solutions were cleaned-up by C18 solid-phase extraction (SPE) by washing with 25% methanol and eluting with 100% methanol.”

Tissue extract preparation accordingly may require a complex series of time-consuming solvation and evaporation steps, and in the case of MBA extract preparation also requires using a highly flammable diethyl ether solvent. While considerable attention has been paid to improving the assays used for analyzing shellfish extracts, little thought appears to have been given to first improving the extract preparation process itself. In particular, by combining a less pure but more easily, more safely or more rapidly prepared tissue extract with one or more complementary assays having compensatory improved accuracy, selectivity or overall BT coverage, faster and potentially much more useful BTX detection can be attained.

An improved complementary assay may be performed using recombinant monoclonal brevetoxin antibodies (BTX-rAbs), rather than the polyclonal antibodies (pAbs) currently employed in ELISA assays for BTX detection. Doing so can be beneficial even if the one or more complementary assays require increased initial investment or higher per-test charges.

Accordingly, in one aspect the present invention provides a device for detecting brevetoxin levels in a sample (e.g., water, urine, food, bivalves and other organisms), the device comprising a test plate comprising a support bearing one or one or more conjugated BTX-rAbs that are cross-reactive with one or more brevetoxin antigens, the conjugated BTX-rAbs being bound to the support or bound to particles that can migrate along the support.

In another aspect the present invention provides a device for detecting brevetoxin levels in a sample, the device comprising a test plate comprising a support bearing i) a sample comprising one or more brevetoxin compounds in a solvent and ii) one or more conjugated brevetoxin antigens or one or more conjugated BTX-rAbs that are cross-reactive with one or more of the brevetoxin antigens, the conjugated brevetoxin antigens or conjugated BTX-rAbs being bound to the support or to bound to particles that can migrate along the support.

In another aspect the present invention provides a system for assessing brevetoxin levels in a sample, the system comprising a test plate comprising a support bearing i) a sample comprising one or more brevetoxin compounds in a solvent and ii) one or more conjugated brevetoxin antigens or one or more conjugated BTX-rAbs that are cross-reactive with one or more of the brevetoxin antigens, the conjugated brevetoxin antigens or conjugated BTX-rAbs being bound to the support or bound to particles that can migrate along the support; and a detector configured to measure concentrations or threshold levels of one or more brevetoxin compounds in such sample based on the extent to which such compounds become or do not become bound to such conjugated brevetoxin antigens or to such conjugated BTX-rAbs.

In yet another aspect the present invention provides a system for assessing brevetoxin levels in a sample, the system comprising a test plate comprising a support bearing i) a sample comprising one or more brevetoxin compounds in a solvent and ii) one or more conjugated brevetoxin antigens or one or more conjugated BTX-rAbs that are cross-reactive with one or more of the brevetoxin antigens, the conjugated brevetoxin antigens or conjugated BTX-rAbs being bound to the support or bound to particles that can migrate along the support; a detector configured to measure concentrations or threshold levels of one or more brevetoxin compounds in such sample based on the extent to which such compounds become or do not become bound to such conjugated brevetoxin antigens or to such conjugated BTX-rAbs; a processor; storage for a set of sample locations, sample dates and measured brevetoxin compound concentrations for such samples; and an engine for detecting whether such concentrations exceed threshold values for any one or more such brevetoxin compounds.

In another aspect the present invention provides a method for assessing brevetoxin levels in a sample, the method comprising obtaining a solvent extract of pulverized tissue, contacting a test plate with the solvent extract, the test plate comprising a support bearing one or more conjugated brevetoxin antigens or one or more conjugated BTX-rAbs that are cross-reactive with one or more of the brevetoxin antigens, the conjugated brevetoxin antigens or conjugated BTX-rAbs being bound to the support or bound to particles that can migrate along the support; obtaining one or more measurements or comparisons to a standard of the concentration of one or more target brevetoxin antigens in such solvent extract. In a preferred embodiment the tissue is bivalve tissue and the method includes a step of permitting or not permitting the harvesting, sale, relocation or depuration of such bivalves based on such measurements or comparisons.

In another aspect, the present invention provides a method for removing brevetoxins from brevetoxin-contaminated bivalves, the method comprising:
a) submerging the contaminated bivalves in water circulating through a toxin removal recirculation system;
b) removing toxins released by the contaminated bivalves by recirculating the water through the toxin removal recirculation system to provide less contaminated bivalves;
c) contacting the above-described test plate with a solvent extract of pulverized tissue from such less contaminated bivalves to determine their remaining brevetoxin contamination; and
d) permitting or not permitting the harvesting, sale, relocation or depuration of such less contaminated bivalves based on such remaining brevetoxin contamination.

In one aspect, the sample is a liquid sample containing cells that may be lysed by freezing, sonication, or use of a blender to permit assessment of the total (intra and extracellular) BTX content of the sample. In another aspect, the extracellular and intracellular measurement methods are performed in sequence.

An especially desirable embodiment of the disclosed device, the system and method will permit testing to be performed on board ship or at dockside. A further especially desirable embodiment of the disclosed device, the system and method will permit testing to be performed on a crude extract obtained using solvent extraction and without requiring solvent evaporation.

### BRIEF DESCRIPTION OF THE DRAWING

The disclosed subject matter may be more completely understood in consideration of the following detailed description of various embodiments in connection with the accompanying Drawing figures, in which:

FIG. 1 is a schematic view of a system for assessing and storing BTX levels;

FIG. 2 is a block diagram of a system for sampling and assessing BTX levels in bivalves, and for monitoring depuration; and

FIG. 3 is a flowchart of a BTX assay.

FIG. 4 is a block diagram of a BTX assay device in use.

Like reference symbols in the various figures of the Drawing indicate like elements. The elements in the Drawing are not to scale.

### DETAILED DESCRIPTION

BTXs are lipid-soluble cyclic polyether compounds currently thought to include more than 10 parent compounds as well as a number of analogs and metabolites responsible for NSP. There are two distinct structural backbones for BTXs, but both are characterized structurally as relatively linear with a bend mid-molecule as shown below:

[chemical structure]

A-type backbone BTXs presently include three known parent compounds and six known metabolites. B-type backbone BTXs presently include 6 parent toxins and 14 metabolites. These compounds are shown below in Table 1, along with an indication of whether or not they are cross-reactive with the only commercially available ELISA assay kit approved for use in the U.S. to assess BTX levels in mollusks:

TABLE 1

| A Toxins | CR* | B Toxins | CR | B Toxins | CR |
|---|---|---|---|---|---|
| BTX-1 | N | BTX-2 | Y | BTX-B3 | — |
| BTX-7 | — | BTX-3 | Y | BTX-B4 | N |
| BTX-10 | — | BTX-5 | Y | BTX-B5 | Y |
| BTX-A | — | BTX-6 | N | S-deoxyBTX-B2 | — |
| BTX-A1 | — | BTX-8 | — | cysteinylglycine-PbTx-B | — |
| BTX-A2 | — | BTX-9 | Y | g-glytamylcysteine-PbTx-B | — |
| S-deoxyBTX-A1 | — | BTX-B | — | N-hexadecanoyl-cysteine-PbTx-B | — |
| S-deoxyBTX-A2 | — | BTX-B1 | — | N-hexadecanoyl-cysteine-PbTx-B-SO | — |
| BTX-A5 | — | BTX-B2 | Y | N-tetradecanoylcysteine-cysteine-PbTx-B | — |
| | | S-deoxyBTX-B2 | — | N-tetradecanoylcysteine-cysteine-PbTx-B-SO | — |

*CR = cross reactive with ELISA as evaluated using MARBIONC Development Group, LLC polyclonal antibody ELISA assay.
— = Not evaluated with ELISA.

Parent compounds BTX-1 (an A-type BT) and BTX-2 (a B-type BT) are considered the most toxic of all BTXs. BTX-1 and BTX-2 have different backbones and share a common unsaturated aldehyde tail region. BTX-3 is the most prevalent BTX found in shellfish and is the reduced form of BTX-2, sharing a common B-type backbone but with a different tail region. The cited ELISA kit is available from MARBIONC Development Group, LLC, and may be used to evaluate BTX levels in oysters, hard clams, and sunray venus clams. It employs an in vitro assay based on an anti-BTX polyclonal antibody (pAb). The selected pAb has a high affinity for several but not all BTXs.

Although developed in the 1960's, the MBA is still considered the gold standard for shellfish toxicity assessment, and continues to be required for the reopening of bivalve beds in Florida and other areas after regulatory closures. The MBA has many deficiencies that result in a lack of timely information needed to approve reopening of closed shellfish harvest areas. The MBA does not actually measure BT levels, as it is not calibrated with known concentrations of BTXs; it instead provides a non-specific measure of the relative toxicity of a shellfish extract (viz., the number of units necessary to cause mortality in a portion of the mice), without determining the specific toxins responsible for the observed mouse mortality. This deficiency can lead to false positive results, something the bivalve farming community believes may happen with some frequency. For example, MBA results sometimes fluctuate significantly over time while ELISA results from the same samples remain steady or decline. Bivalve farmers may view these divergent results as confirming false positive results from the MBA. Also, MBA sample throughput is limited (often to two samples/week) by the availability of suitable mice. This limitation may leave a bivalve farmer unable to get its product tested, even when the farmer is confident the product is safe. The MBA also requires testing live animals in a research facility approved, certified and inspected by the USDA, making it a costly method to sustain.

ELISA is an important alternative to the MBA due to its relatively rapid assessment capabilities, low detection levels, inexpensive costs, satisfactory use by less skilled scientists, and the elimination of the need for animal test subjects. While ELISA results, if favorable, provide improved testing efficiency compared to the MBA, many bivalve growers regard the current ELISA format and application as having significant limitations. For example, ELISA requires a specialized laboratory instrument for reading the sample assays, and a minimum of two days of critical time to receive results (one day for collection and same-day/rush transport to the lab, and one day for analysis). As noted above, the current approved ELISA kit has not been found to detect the presence of A-type backbone BTXs or the B-type toxins BTX-6 and BTX-B4, and cross reactivity for only a few B-type backbone metabolites has been assessed. Presently, the ELISA BTX threshold regarded as “safe” in clams and oysters is equivalent to only 50% of the regulatory ≤20 MU limit. This regulatory buffer is regarded by many bivalve farmers as a hardship that prevents them from harvesting what may well be safe, wholesome product due to an analytical dysfunction in the ability to correlate ELISA and MBA results above the ELISA BTX threshold, and due to a need to obtain MBA results before reopening an SHA.

Much of the present disclosure is, in the interest of expediency and by way of example, focused on bivalve samples and bivalve harvesting. However, those skilled in the art will based on reading this disclosure understand that the principles, devices, systems, and methods described herein may be applied for the evaluation of BTX levels in a variety of samples other than bivalve tissue extracts. The disclosed device and method accordingly may be used with a variety of samples, including but not limited to water, urine and other fluids; tissue from bivalves, fish, marine mammals and other mammalian organisms; marine or terrestrial crops; other foods; and other liquid, gel, semisolid or solid substances. The samples may each be taken from a single location, or may be a combined sample taken from a plurality of locations that can be reexamined individually or in smaller groups if the combined sample provides a positive result. Water samples may for example be from seawater, fresh water, desalinated water, drinking water, swimming water, water for or from aquaculture, drainage water, sewer water, or water obtained from any other source. Tissue samples may for example be from land or marine organisms including captured, cultured, reared (including aquaculture-reared) animals or organisms including mammals, amphibians, reptiles, fish and shellfish, or from any other animals or organisms obtained using any other suitable technique. Further discussion of the use of the disclosed device and method may be found in U.S. Provisional Application No. 62/934,351 filed Nov. 12, 2019, the entire disclosure of which is incorporated herein by reference.

As discussed above, the present invention employs an improved complementary assay that uses recombinant brevetoxin antibodies (rAbs). Selection and development of an rAbs assay can take place in vitro using synthetic genes and a targeted selection strategy employing a fragment antibody (Fab) to bind to a chosen antigen. This provides several advantages over a pAbs assay. Animals are not required, thereby shortening development time, and production can occur in the bacteria E. coli, providing a continuous source of Abs (unlike in vivo derived pAbs). Use of a Fab for antigen binding can also provide improved specificity and high affinity, factors that are especially useful in a point of use (POU) biosensor. An assay based on appropriate rAbs can also optimize the binding of both A- and B-type BTXs, a distinct advantage over the current pAbs ELISA assay. Without being bound by theory, rAbs may provide enhanced cross-reactivity and thus enhanced detection for either A- or B-type backbone BTXs, allowing for a more thorough and sensitive screening method for bivalves.

Referring now to FIG. 1, representative system 100 for processing and measuring brevetoxin(s) concentrations is shown. Automated instrument 101 supports a microtiter plate 102 containing sample wells 104 and positioned atop movable stage 106. Once loaded with samples, stage 106 passes into housing 108 where incubation, washing, antibody addition and absorbance measurement steps are performed. Control of the operation of instrument 101 can be performed and the results for selected samples can be displayed using touch panel 110. The results may be stored and processed for analysis using a suitable engine. The term engine as used herein is defined as a real-world device, component, or arrangement of components implemented using hardware, such as by an application specific integrated circuit (ASIC) or field-programmable gate array (FPGA), for example, or as a combination of hardware and software, such as by a microprocessor system and a set of program instructions that adapt the engine to implement a desired functionality, which while being executed may transform a microprocessor system into a special-purpose device. The above-mentioned measurement results may be stored within instrument 101, stored in a nearby or networked separate storage location (not shown in FIG. 1) or remotely stored using for example cloud storage facility 112. The above-mentioned engine may reside within instrument 101, within a nearby or networked separate processing device (not shown in FIG. 1) or may be remotely processed for analysis using for example remote engine and processor 114.

A block diagram 200 of steps that may be employed in the disclosed method is shown in FIG. 2. A sample is collected 202 from a bivalve, and optionally processed 204 to obtain a release of brevetoxin into the sampling solution. The sample is optionally transported 206 to a measurement instrument where brevetoxin concentration measurements are obtained 208 for one or more brevetoxins (as discussed in more detail below). The measurements are stored 210 along with other previously or subsequently stored measurements for bivalves from the same population or region, using for example onboard, nearby, networked or cloud storage. The stored measurements are analyzed 212 using for example onboard, nearby, networked or cloud computing. Based on analysis 212, an alternative 214 is followed, namely to close or detoxify 216 or otherwise delay any fishing, transportation, or sale of bivalve mollusks for a time period, and to instead continue monitoring or depuration, or to permit fishing 218, transportation, or sale of the bivalve mollusks.

A block diagram 300 demonstrating the general principles of the water BTX content detector is shown in FIG. 3. A biosensor element, e.g., rAbs, is immobilized 302 and treated or otherwise applied to the surface of a sensor or transducer 304. The selected antibodies may be immobilized on sensor or transducer 304 by a measure such as absorption, entrapment, covalent coupling, affinity, the use of binding proteins, chemical binding to polypeptide strands, or any other binding method. The binding method may be selected and configured such that exposure of the paratope is optimized after binding to permit unimpaired antibody-antigen complex formation. In embodiments, the rAbs may be self-immobilizing.

A target analyte, e.g., a BTX toxin, interacts with the biosensor element 306 to cause analyte recognition 308 and conversion to a signal by the transducer 310. The resulting signal may depend upon the nature of the biosensor employed (e.g., an electrochemical, electronic, optical, piezoelectric, gravimetric, pyroelectric or other suitable biosensor). The signal is amplified 312 and converted to a readout 314, e.g., converted to a numerical output using an algorithm, and the readout is displayed 316. Depending on the needs of the user and the system, the output data 316 may be used as feedback to improve the sensor and improve the selection of the biosensor components 302. Results may also be cross-analyzed for verification with a standard or results from another well-known assay, such as ELISA.

A block diagram 400 demonstrating a detection process for the disclosed biosensor is shown in FIG. 4. A transducer 402 may be prepared for biosensor function by applying a surface treatment 404 comprising immobilized rAbs 406. Brevetoxins 408 interact with the immobilized biomolecules 406 producing a bio signal 410 which is converted into an electrical signal 412 by the transducer 402. Following amplification and conversion, the signal 410, 412 results in a display output 414. Transducer 402 may generally use physicochemical processes to transform the bio signal 410. For example, transducer 402 may be optical, piezoelectric, electrochemical, electrochemiluminescent, etc.

The disclosed analysis may be performed using a variety of engines, each of which is constructed, programmed, configured, or otherwise adapted, to autonomously carry out a function or set of functions. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine may be realized in a variety of physically realizable configurations, and should generally not be limited to any particular implementation discussed or exemplified herein, unless such limitations are expressly called out. In addition, an engine can itself be composed of more than one sub-engine, each of which can be regarded as an engine in its own right. An engine or a variety of engines may correspond to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically discussed herein.

Various embodiments of the disclosed system, and the corresponding methods of configuring and operating the disclosed system, may be performed using cloud computing, client-server, or other networked environments, or any combination thereof. The components of the system can be located in a singular “cloud” or network, or spread among many clouds or networks. End-user knowledge of the physical location and configuration of components of the system is not required.

As will be readily understood by one of skill in the art, the disclosed system may be implemented using at least one processor and operably coupled memory. The processor can be any programmable device that accepts digital data as input, is configured to process the input according to instructions or algorithms, and provides results as outputs. In an embodiment, a processor can be a central processing unit (CPU) configured to carry out the instructions of a computer program. A processor is therefore configured to perform at least basic arithmetical, logical, and input/output operations.

Memory operably coupled to the processor can include volatile or non-volatile memory as required by the coupled processor to not only provide space to execute the instructions or algorithms, but to provide the space to store the instructions themselves. In embodiments, volatile memory can include random access memory (RAM), dynamic random-access memory (DRAM), or static random-access memory (SRAM), for example. In embodiments, non-volatile memory can include read-only memory, flash memory, ferroelectric RAM, hard disk, floppy disk, magnetic tape, or optical disc storage, for example. The foregoing lists in no way limits the type of memory that can be used, as these embodiments are given only by way of example and are not intended to limit the scope of the disclosed system. The disclosed storage component generally includes electronic storage for data concerning the days and optionally the times at which samples have been taken and a name, number or other identifier for the mare or mares from which the samples were obtained. In an embodiment, the disclosed storage may be a general-purpose database management storage system (DBMS) or relational DBMS as implemented by, for example, Oracle, IBM DB2, Microsoft SQL Server, PostgreSQL, MySQL, SQLite, Linux, or Unix solutions, and for which SQL calls may be utilized for storage and retrieval. In another embodiment, the disclosed storage, engine or both may employ a cloud computing service such as the Amazon Web Services (AWS) cloud computing service.

The invention is further illustrated in the following non-limiting examples, in which all parts and percentages are by weight unless otherwise indicated.

### Example 1

### HuCAL BTX Antibody Selection

Recombinant antibody rAb fragments from the HuCAL PLATINUM™ phage library (Bio-Rad Laboratories, Inc.) are used to identify BTX-rAbs for use in a POU test kit for monitoring BTXs in shellfish during and after red tide blooms. Fragments that may bind to both A- and B-type BTX backbones are selected using guided selection for epitope recognition and in vitro testing, enabling greater flexibility for Ab generation than conventional methods based on animal immunization. Guided selection strategies involve either blocking steps or the use or two of more antigens, and may be used for the isolation of epitope-specific Abs or for Abs that recognize shared epitopes on different antigens. B-type brevetoxin-conjugate, immobilized on a solid support, is employed for a first round screening. Abs identified by the first round of screening are then presented to an A-type brevetoxin-conjugate, also immobilized on a solid support. One, two, three, or more rounds of additional screening may be performed to enrich the Ab library for specific BTX binding. The activity and specificity of the BTX-rAbs may be tested by a quality control (QC) ELISA, in which the BTX-rAbs are tested on non-related standard antigens and on positive and negative control antigens. The strategic use of two antigens enables the method to identify BTX-rAbs with BTX-3 (associated with B-type brevetoxin) used for the first round of screening followed by BTX-1 (associated with A-type brevetoxin) for the second round.

As discussed above in connection with Table 1, BTX-3 is the reduced form of BTX-2, sharing a common B-type backbone but with a different tail region, and BTX-2 and BTX-1 have different backbones and share a common unsaturated aldehyde tail region. During BTX-rAb screening, the common tail region may dominate the enrichment process, yielding more rAbs targeting that region over the desired backbone regions. Screening first with BTX-3, then BTX-1, facilitates identification of BTX-rAbs that will recognize the A-ring lactone region, the rigid bend region, or the K-ring side chain, all similar regions of the A- and B-type backbones, as well as facilitating identification of BTX-rAbs that cross-react with all BTXs.

### Example 2

### K. Brevis Uptake, Depuration Exposure and Sample Collection

The middle neck clam (M. mercenaria) is economically important to shellfish growers throughout various regions of the US and one of the species often implicated in neurotoxic shellfish poisoning (NSP). Clams are subjected to a constant exposure of cultured K. brevis at a concentration of 20,000 cells/L for 5 days followed by a 7-day clean filtered seawater depuration period to determine their ability to purge themselves of brevetoxins under depuration conditions. The claims are approximately one year old and farm-raised, and are evenly distributed among 9 raceway tanks (n=125 clams per tank), each with a working volume of 0.55 m³ (552 L). The tanks are part of a proven, large-scale, zero-discharge, system designed and used to conduct exposure experiments concerning the effect of the Deepwater Horizon spill on aquatic animals. Target flow rates are monitored and adjusted daily as needed to maintain target cell counts of K. brevis. Among the 9-replicate tanks, 3 are designated as control (no exposure to K. brevis) and 6 are dedicated to exposure.

The clams are fed a commercially available marine algal diet formulated specifically for shellfish at a target of 100-150 cells/mL twice daily. Environmental parameters are maintained according to standard protocols associated with optimal rearing conditions for the selected species. Water quality, including temperature (26° C.), salinity (30 ppt), dissolved oxygen, and pH are monitored daily as are the water chemistry parameters ammonia and nitrite. Throughout the test, the requirements normally employed for bacterial removal from shellfish (see FDACS, Division of Aquaculture Rule 5L-1.015 titled “Depuration and Wet Storage Facility Operations” and the Comprehensive Shellfish Control Code) are also followed for this BTX depuration process.

K. brevis (Wilson strains) is cultured at 24° C. on a 12 h light: 12 h dark photoperiod (50-60 μmol m-2 s-1) at 32-34 salinity in a modified L1 media with NH15 vitamins. Growth is monitored by counting a 1:10 dilution of culture in Z-Pak reagent using a Beckman Z-series Coulter Counter with aperture size between 10˜30 μm, according to the manufacturer's instructions. The exposure solution is prepared fresh each day by diluting fresh cultured K. brevis with filtered seawater to create the targeted concentration using two 1425 L mixing reservoirs, each equipped with a rotating propeller operated at very low rpms to keep the cells suspended for a homogeneous distribution. During the exposure events, K. brevis-free seawater (for controls) or fresh exposure solutions are constantly cycled through the exposure tanks to the water filtration system to maintain required tank turnover rates. The 2017 National Shellfish Sanitation Protocol (NSSP) Model Ordinance (e.g., Section 2 Chapter XV) and FDACS, Division of Aquaculture Rule 5L-1.015 protocols for the construction and maintenance of bacterial depuration and wet storage systems are also followed for this BTX depuration process.

To confirm the efficacy of the BTX-rAbs as a monitoring tool in screening shellfish for BTXs, composite samples of 18-clams each are collected from 3-exposure and 3-control tanks daily during the 5-day uptake exposure and every other day during the 7-day depuration period. This temporal sampling scheme generates tissues with a range of BTX concentrations; increasing BTX levels during accumulation and decreasing levels during depuration. After sampling, the clams are cleaned, shucked, drained, homogenized, extracted (see methods below), labeled and stored at −20° C. until analysis. In order to correlate the assays with the NSSP approved methods for BTX screening in clams, extraction for the MBA follows the American Public Health Association protocol. For ELISAs (BTX-rAbs and MARBIONC) and LC/MS-MS analyses, the acetone-methanol extraction protocol is used. Water samples from the exposure tanks are assessed for K. brevis cell counts may be determined by iodine staining and microscopy.

### Example 3

### Validation of HuCAL BTX-rAbs

To verify the specificity of the Example 1 BTX-rAbs to detect both the A- and B-type BTXs in the clam matrix, a competitive ELISA is done using control and exposed clams. Clams are spiked with known concentrations of A- and B-type parent and shellfish metabolite compounds. For validating and assessing the accuracy of the BTX-rAbs ELISA, spike and recovery, and linearity of dilution experiments are done. The spike and recovery determine whether toxin detection is affected by a difference between the biological samples (i.e., clam matrix) and standard diluent. Success is assessed based on 80-120% recovery of the toxins. Linearity of dilution assesses the predictability of the spike on natural recovery for known dilution factors in the desired assay range. Acceptable sample dilutions fall in the 20-80% standard range. Samples from the exposure study are then assessed for BTXs using the selected BTX-rAbs fragments in an ELISA analysis.

### Example 4

### Tissue Analysis

Clam samples generated from the Example 2 exposure are analyzed to compare and correlate the BTXs levels detected by the Example 3 BTX-rAbs ELISA analysis, alongside data generated using the above-mentioned MARBIONC ELISA kit, LC/MS-MS and toxicity as determined using the MBA. Clam tissue samples are homogenized for use in all subsequent analyses. The assays performed include:

Liquid chromatography/mass spectrometry-mass spectrometry (LS/MS-MS). Sub-samples of homogenized clam tissue are acetone:methanol extracted and passed through a conditioned C18 SPE column and eluted with methanol using the method described in Plakas et al., Brevetoxin Metabolism and Elimination in the Eastern Oyster (Crassostrea virginica) after Controlled Exposures to Karenia brevis, Toxicon 2004, 44 (6), 677-685. Brevetoxins are structurally confirmed and quantitated with available BTX standards, using a Thermo TSQ Triple Quad LC-MS/MS with electrospray ionization (ESI).

ELISA. From the homogenized clam tissue samples, tissue is extracted following the MARBIONC ELISA kit protocols and analyzed with the MARBIONC ELISA kit following the manufacturer's protocol. BTX-rAbs ELISA protocols are established by quality assurance and quality control (QAQC) based on spike recovery, linear response, and cross-reactivity using the same extract.

Mouse bioassay assay (MBA). A 100 g sub-sample of the homogenized tissue is frozen for MBA testing. The MBA is conducted by a NSSP approved laboratory following the APHA protocol specified for regulatory acceptability.

### Example 5

### Data Comparison and Correlation

To compare and correlate data from the analytical methods performed, the same sample homogenized tissues are analyzed using each protocol. Uptake and depuration dynamics are determined. Data is correlated and evaluated using linear regression, variance and Spearman rank correlation analyses and by using R statistics.

In addition to samples from bivalves, those of skill in the art will understand that the same or similar devices, systems, methods, and principles will also serve for the preparation and testing of other tissue samples and tissue extracts. Exemplary embodiments include sampling and evaluating other marine and terrestrial organisms which may be exposed or contaminated with BTX from environmental or dietary sources.

### Example 6

### Water Testing for Extracellular Toxin Levels

In situ biomarker test kit for onsite testing of water for the presence of brevetoxins. Embodiments of the present disclosure provide for testing at the point of collection, and may, for example, be performed at a shipboard or shoreline location where the sample is drawn. Though the present example focuses on water samples, those skilled in the art will understand that the same or similar devices, systems, methods, and principles will function to test other liquid samples, including but not limited to urine, saliva, and other secretions produced by live organisms which may be subject to BTX contamination by environmental or dietary sources.

Test performance may generally be improved by removal of particulates prior to testing, such as by simple filtration. Samples may be pretreated at the time of collection to minimize absorption by the sample container. Samples may be tested using a dipstick, prepared well or slides, a biosensor etc. pre-treated with the disclosed BTX sensitive rAbs.

### Example 7

### Water Testing for Total (Intracellular and Extracellular) Toxin Levels

Samples may be flash-frozen, sonicated, mechanically agitated, etc. prior to testing to rupture intact K. brevis cells and release contained BTXs. Samples thus prepared will yield a result inclusive of the total BTX level.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A device for detecting brevetoxin levels in a sample, the device comprising:
a test plate comprising a support bearing one or one or more conjugated recombinant monoclonal brevetoxin antibodies (BTX-rAbs) that are cross-reactive with one or more brevetoxin antigens, the conjugated BTX-rAbs being bound to the support or bound to particles that can migrate along the support.

2. The device of claim 1, wherein the support also bears a sample comprising one or more brevetoxin compounds in a solvent.

3. The device of claim 2, wherein the sample is taken from a single location.

4. The device of claim 2, wherein the sample is a combined sample taken from a plurality of locations.

5. The device of claim 2, wherein the sample is taken and tested aboard a watercraft.

6. The device of claim 2, wherein the sample is a water sample.

7. The device of claim 6, wherein the sample comprises ocean water.

8. The device of claim 6, wherein the sample comprises fresh water.

9. The device of claim 6, wherein the sample comprises aquaculture water.

10. The device of claim 6, wherein the sample comprises depuration water.

11. The device of claim 2, wherein the sample comprises urine.

12. The device of claim 11, wherein the urine is from a human being.

13. The device of claim 11, wherein the urine is from a marine organism.

14. The device of claim 2, wherein the sample comprises fish tissue.

15. The device of claim 2, wherein the sample comprises shellfish tissue.

16. The device of claim 15, wherein the sample comprises bivalve tissue.

17. The device of claim 15, wherein the sample comprises clam tissue.

18. The device of claim 15, wherein the sample comprises oyster tissue.

19. The device of claim 15, wherein the sample comprises mussel tissue.

20. The device of claim 15, wherein the sample comprises scallop tissue.

21. The device of claim 2, wherein the sample comprises marine plant tissue.

22. The device of claim 2, wherein the sample comprises a food.

23. The device of claim 2, wherein the BTX-rAbs are cross-reactive with two or more brevetoxin antigens.

24. The device of claim 2, wherein the BTX-rAbs are cross-reactive with three or more brevetoxin antigens.

25. The device of claim 2, wherein the BTX-rAbs are cross-reactive with antigens of both A-type backbone and B-type backbone brevetoxin compounds or metabolite brevetoxin species.

26. The device of claim 2, wherein the BTX-rAbs are cross-reactive with antigens of both BTX-1 and BTX-2.

27. The device of claim 2, wherein the BTX-rAbs are cross-reactive with antigens of each of BTX-1, BTX-2 and BTX-3.

28. A system for assessing brevetoxin levels in a sample, the system comprising:
a test plate comprising a support bearing i) a sample comprising one or more brevetoxin compounds in a solvent and ii) one or more conjugated brevetoxin antigens or one or more conjugated recombinant monoclonal brevetoxin antibodies (BTX-rAbs) that are cross-reactive with one or more of the brevetoxin antigens, the conjugated brevetoxin antigens or conjugated BTX-rAbs being bound to the support or bound to particles that can migrate along the support; and
a detector configured to measure concentrations or threshold levels of one or more brevetoxin compounds in such sample based on the extent to which such compounds become or do not become bound to such conjugated brevetoxin antigens or to such conjugated BTX-rAbs.

29. The system of claim 28, further comprising:
a processor;
storage for a set of sample source locations, sample dates and measured brevetoxin compound concentrations for such samples; and
an engine for detecting whether such concentrations exceed threshold values for any one or more such brevetoxin compounds.

30. The system of claim 28, wherein the sample is a crude extract obtained using solvent extraction and without requiring solvent evaporation.

31. A method for assessing brevetoxin levels in a sample, the method comprising the steps of:
obtaining a solvent extract of pulverized tissue from an organism;
contacting a test plate with the solvent extract, the test plate comprising a support bearing one or more conjugated brevetoxin antigens or one or more conjugated recombinant monoclonal brevetoxin antibodies (BTX-rAbs) that are cross-reactive with one or more of the brevetoxin antigens, the conjugated brevetoxin antigens or conjugated BTX-rAbs being bound to the support or bound to particles that can migrate along the support;
obtaining one or more measurements or comparisons to a standard of the concentration of one or more target brevetoxin antigens in such solvent extract; and
permitting or not permitting the harvesting, sale, or relocation of such organisms based on such measurements or comparisons.

32. The method of claim 31, further comprising removing brevetoxins from brevetoxin-contaminated bivalves by:
a) submerging the contaminated bivalves in water circulating through a toxin removal recirculation system;
b) removing toxins released by the contaminated bivalves by recirculating the water through the toxin removal recirculation system to provide less contaminated bivalves;
c) contacting such test plate with a solvent extract of pulverized tissue from such less contaminated bivalves to determine their remaining brevetoxin contamination; and
d) permitting or not permitting the harvesting, sale, relocation or depuration of such less contaminated bivalves based on such remaining brevetoxin contamination.
